(19) United States
(12) Patent Application Publication
(10) Pub. No.: US 2011/0015669 A1
(43) Pub. Date: Jan. 20, 2011

(54) Forceps

(76) Inventor: Borja F. Corcosteugi, Barcelona (ES)

Correspondence Address: LAW OFFICES OF JERRY A. SCHULMAN, 1S376 SUMMIT AVENUE, COURT C, OAKBROOK TERRACE, IL, 60181, US

(21) Appl. No.: 12/265,276

(22) Filed: Nov. 5, 2008

Related U.S. Application Data
(60) Provisional application No. 60/986,491, filed on Nov. 8, 2007.

(51) Int. Cl. A61B 17/28

(52) U.S. Cl. 606/207

(57) ABSTRACT

A microsurgical forceps has a pair of opposed jaws which are configured to create a window through which a user can view tissue underlying the jaws. The window and the tapered lateral profiles of the jaws increase the field of view available to a user.

This application claims priority from U.S. Patent Application Ser. No. 60/986,491, filed Nov. 8, 2007, which is incorporated herein in its entirety by reference.

### FIELD OF THE INVENTION

This invention relates generally to surgical instruments designed for use in eye surgery and, more particularly, to a forceps used to remove fibrous material from the eye and to lift the tissue to be removed in the manner of a pick or spatula.

### BACKGROUND OF THE INVENTION

Certain ophthalmological surgical procedures require the peeling or delamination of the retinal ILM. A description of the various surgical techniques requiring delamination may be found in the second edition of Vitreous Microsurgery by Steve Charles, Williams and Wilkins, 1987, particularly at pages 120-121, 222, 163 and 164. Charles describes the use of a scissors to effect cuts in the ILM, reducing it to small enough pieces to be removed from the eye.

Surgeons now use blunt dissection techniques, employing instruments such as the VRTIS™ ILM spatula sold by ASICO, LLC, of Westmont Ill. as its Model Nos. AE-2917 and AE-2921. Such a spatula includes a handle to which a shaft is mounted with the shaft terminating in a shaped tip having no sharp edges. The tip is typically contoured and slightly curved to enable manipulation of the spatula along the rear portion of the eye to perform a delamination of the ILM. The blade is shaped for delamination of the ILM and can be manipulated to bring it into a preferred position for delaminating other portions of the ILM.

After removal of the ILM fibrous tissue remaining behind must also be removed. Use of a forceps such as the VRTIS 23 gauge fine and gripping forceps marketed by ASICO, LLC of Westmont, Ill. as its Model AE-4912 can carry out the fiber removal procedure.

The use of picks and forceps to remove portions of the ILM are described in U.S. Pat. No. 6,024,719 (Morris); U.S. Pat. No. 6,210,357 (Morris); and U.S. Patent Application Publication 2006/0116703 (Glaser).

### BRIEF DESCRIPTION OF THE INVENTION

An improved gripping forceps particularly useful in ILM delamination has a jaw geometry which increases the surgeon's field of vision. The jaws are shaped to be used in much the same manner as a pick is used, that is, to engage the periphery of the ILM and to lift it, beginning the delamination and removal process.

While the following describes a preferred embodiment or embodiments of the present invention, it is to be understood that this description is made by way of example only and is not intended to limit the scope of the present invention. It is expected that alterations and further modifications, as well as other and further applications of the principles of the present invention will occur to others skilled in the art to which the invention relates and, while differing from the foregoing, remain within the spirit and scope of the invention as herein described and claimed. Where means-plus-function clauses are used in the claims such language is intended to cover the structures described herein as performing the recited functions and not only structural equivalents but equivalent structures as well. For the purposes of the present disclosure, two structures that perform the same function within an environment described above may be equivalent structures.

### DETAILED DESCRIPTION OF THE INVENTION

These and other aspects of the present invention will best be understood by reference to the accompanying drawings wherein:

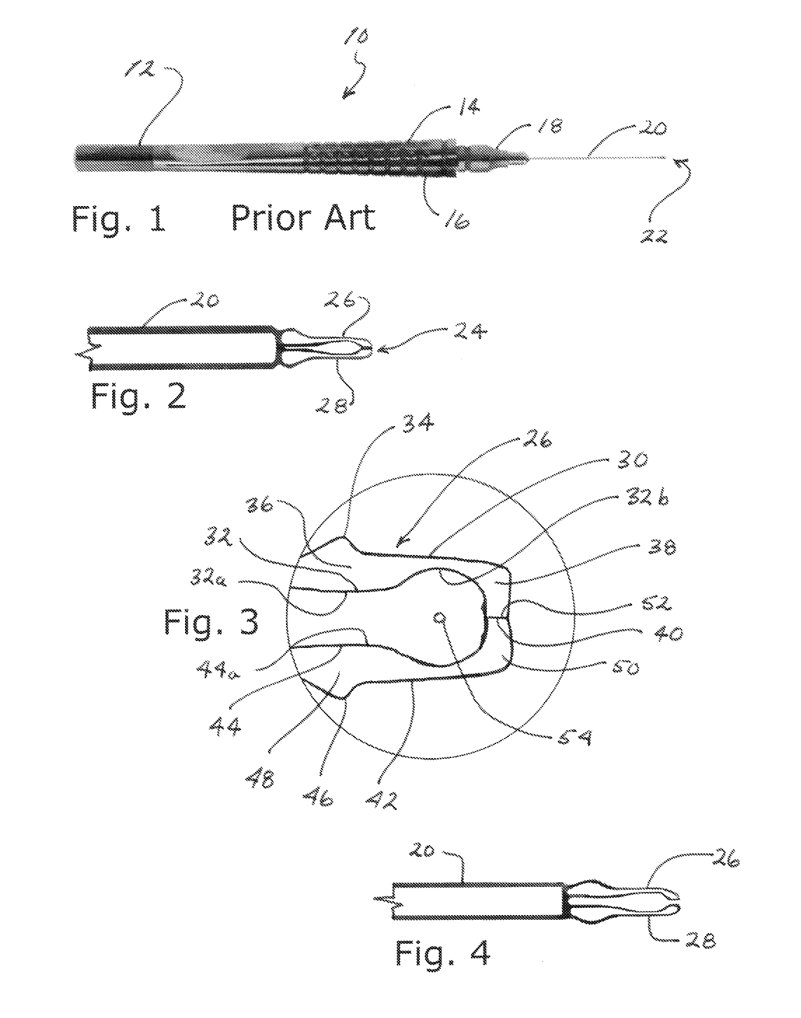

FIG. 1 is a perspective view of a prior art gripping forceps;

FIG. 2 is a partial perspective view of a gripping forceps in the closed position with jaws embodying principles of the present invention;

FIG. 3 is an enlarged detail of a portion of the jaws of FIG. 2;

FIG. 4 is a partial perspective view of the forceps of FIG. 2 with the jaws in a partially opened position;

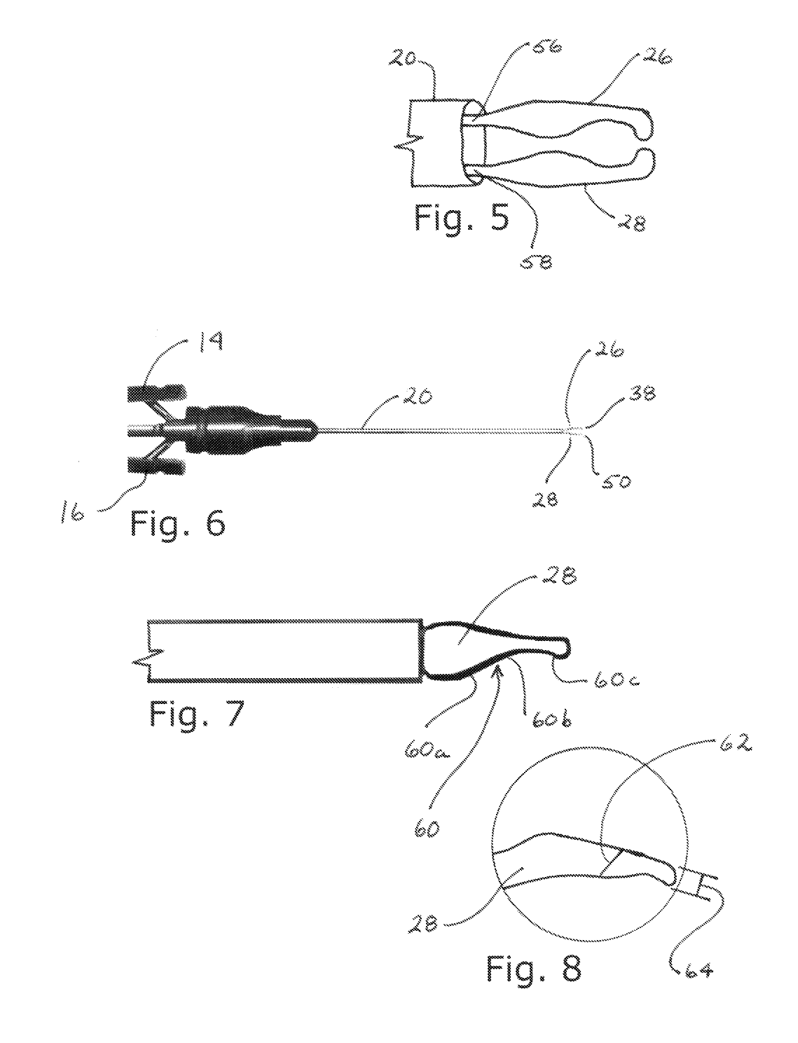

FIG. 5 is an enlarged detail of the jaws of the forceps shown in FIG. 4;

FIG. 6 is a partial perspective view showing the jaws completely open;

FIG. 7 is a partial lateral perspective view of the forceps of FIG. 2 showing the jaws in a closed position; and

FIG. 8 is an enlarged detail of a portion of the jaws shown in FIG. 7.

### DETAILED DESCRIPTION OF THE DRAWINGS

Referring now to FIG. 1, the numeral 10 identifies a prior art gripping forceps having a body 12 to which a pair of operating handles 14, 16 are hingedly mounted. Body 12 terminates in a mount 18 within which a hollow tube 20 is slidably held. A pair of spring steel forceps elements, each terminating in a jaw is disposed within tube 20 and are secured within body 12 so that the elements do not move. The jaws extend from tube 20 and, when tube 20 is withdrawn, are biased to be urged apart from one another. In the example shown, which represents a commonly available and known prior art body and handle assembly, when handles 14, 16 are compressed toward body 12, tube 20 is advanced, engaging ramps formed on the exterior surfaces of the jaws and forcing the forceps elements towards each other to bring the jaws into contact with one another. For purposes of identification in FIG. 1, the numeral 22 indicates generally the jaws as they protrude from tube 20. For purposes of further description, it will be assumed that gripping forceps 10 has had its original forceps elements replaced by forceps elements which terminate in jaws that embody the present invention

Referring now to FIG. 2, a detail of a forceps is shown with each forceps element having a jaw that embodies certain principles of the present invention. As can be seen in FIG. 2, a jaw assembly 24 is shown in a first or closed position. Jaw assembly 24 comprises jaw 26 and jaw 28. For purposes of description, jaw 26 will be described as the left jaw while jaw 28 will be described as the right jaw. It should be understood that the left and right jaws 26, 28 are formed at the distal end of forceps elements such as those described hereinabove with the remainder of the forceps elements disposed within tube 20.

Referring now to FIG. 3, left jaw 26 is shown having an outer surface 30 and an inner surface 32. Outer surface 30 has a ramp 34 formed thereon which extends outwardly from surface 30. A first segment 36 of left jaw 26 extends in a generally longitudinal direction and then terminates in a jaw tip 38 which is formed generally perpendicular to segment 36. Jaw tip 38 terminates at jaw end 40.

In the example shown, right jaw 28 is a mirror image of left jaw 26, having an outer surface 42, an inner surface 44, a ramp 46, a first longitudinally extending segment 48 and a jaw tip 50 terminating at a jaw end 52.

Inner surface 32 has a first segment 32a which extends in a generally longitudinally direction, and a second segment 32b which is arcuate in shape and which curves toward outer surface 30 and which terminates at the point at which jaw tip 38 and 48 are in contact with each other. A similar configuration is seen with segments 42a and 42b of right inner jaw surface 42. The effect of this shaping is to create a “window” or “bight”54 when jaws 26, 28 are fully closed and jaw ends 40, 52 are in contact with one another, through which the surgeon may observe the operating field, an advantage which aids in the location of tissue to be removed and the manipulation of the forceps to grasp this tissue.

In the present example, each jaw tip 38, 50 is approximately 0.40 mm in length and jaw assembly 24, when closed, is approximately 0.60 mm wide when measured from ramp 34 to ramp 46, and is 0.35 mm wide when measured adjacent to said ramps, tapering down to a minimum width of about 0.30 mm across jaw tips 38, 50. Each jaw tip 38, 50 is about 0.15 mm in length, about 0.16 mm in width and about 0.18 mm in thickness.

Referring now to FIG. 4, left and right jaws 26, 28 are shown in a partially opened position. This results when handle 14, 16 are allowed to move apart from one another which, in turn, allows tube 20 to move longitudinally toward body 12, thereby disengaging ramps 26, 28 and allowing the natural tendency of the forceps elements to separate.

An enlarged view of this is shown schematically in FIG. 5 with the withdrawal of tube 20 shown in a somewhat exaggerated illustration to show left forceps element 56 and right forceps element 58. Surfaces 32a, 32b, 42a and 42b allow an even greater field of vision when jaws 26, 28 are partially opened as well.

Referring now to FIG. 6, handles 14, 16 are fully extended and tube 20 is retracted sufficiently to allow left and right jaws 26, 28 to reach their maximum opening. In this position, it should be appreciated that jaw tips 38 and 50 are sufficiently separated to allow them to be manipulated to “hook” one said jaw tip under the outer margin of the ILM, thus providing the forceps with not only a gripping capability but the ability to function as a pick or spatula. When the jaw tips 38, 50 are so used, it is not necessary to introduce a second instrument, namely a pick or spatula, to begin the separation of the ILM and, thereafter, to reinsert the forceps to grip that portion of the ILM and remove it.

FIG. 7 shows a lateral view of forceps 10 with left and right jaws 26, 28 in the fully closed position. It can be seen that the lower surface 56 of right jaw 28 has a first segment 56a which extends generally longitudinally, a second segment 56b which curves upward and away from segment 56a, and a third segment 56c at which the curvature 56b is carried slightly downward. Left jaw 26 is a mirror image of right jaw 28 and has a similarly contoured lower surface. The effect of this configuration is to provide a narrowed profile which provides a larger field of vision for the surgeon.

As seen in FIG. 8, in a preferred embodiment, jaw 28 is about 0.23 mm across at portion 62 and is about 0.18 mm across jaw tip 48 at segment 64.

I claim:

1. A microsurgical forceps, said forceps of the type having first and second opposed jaws moveable from a first, open position toward one another to a second closed position, said forceps comprising:
means for moving said first and second jaws from said open position to said closed position;
at least said first jaw terminating in a jaw end;
said first jaw having a first portion extending from said first jaw end toward said moving means,
said first portion spaced apart from said second jaw to define a window when said first and second jaws are in said closed position, thereby increasing the field of vision of said forceps.

2. The apparatus as recited in claim 1 wherein said first jaw end is formed to extend generally toward said second jaw.

3. The apparatus as recited in claim 2 wherein said second jaw has a second jaw end,
said second jaw end formed to extend generally toward said first jaw end;
said first and second jaw ends contacting one another when said forceps is in said closed position.

4. The apparatus as recited in claim 1 wherein said second jaw has a second jaw end,
said second jaw has a second portion extending from said first second jaw end toward said moving means;
each said jaw having a lateral profile extending from each said jaw end toward said moving means,
each said lateral profile tapering from said moving means toward said jaw end whereby each said lateral profile is smaller in lateral area along said first and second jaw portions.

5. A microsurgical forceps, said forceps of the type having first and second opposed jaws moveable from a first, open position toward one another to a second closed position, said forceps comprising:
means for moving said first and second jaws from said open position to said closed position;
said first jaw terminating in a first jaw end;
said first jaw having a first jaw portion extending from said first jaw end toward said moving means;
said second jaw terminating in a second jaw end;
said second jaw having a second jaw portion extending from said second jaw end toward said moving means,
said first and second jaw portions spaced apart one from another to form a window when said first and second jaws are in said closed position, thereby increasing the field of vision of said forceps.

5. The apparatus as recited in claim 4 wherein said first jaw portion has a first outer surface and a first inner surface substantially coextensive with and opposed to said first outer surface;
said second jaw portion has a second outer surface and a second inner surface substantially coextensive with and opposed to said second outer surface,
said first inner surface curved toward said first outer surface proximate said first jaw end to form a first bight;
said second inner surface curved toward said second outer surface proximate said second jaw end to form a second bight,
said first and second bights substantially registering one with the other when said first and second jaws are moved to said closed position thereby defining a window thereby increasing the field of vision of said forceps.

6. The apparatus as recited in claim 5 wherein said first jaw end is formed to extend generally toward said second jaw end.

7. The apparatus as recited in claim 6 wherein said first and second jaw ends contact one another when said forceps is in said closed position.

8. The apparatus as recited in claim 1 wherein each said jaw has a lateral profile extending from each said jaw end toward said moving means,
each said lateral profile tapering from said moving means toward one said jaw end whereby each said lateral profile is smaller in lateral area along said first and second jaw portions.
